(19) United States
(12) Patent Application Publication
(10) Pub. No.: US 2025/0250340 A1
(43) Pub. Date: Aug. 7, 2025

(54) METHOD TO PURIFY AN ANTIBODY COMPOSITION

(71) Applicant: DR. REDDY’S LABORATORIES LIMITED, Hyderabad (IN)

(72) Inventors: Anitha PUPPALA, Kukatpally Hyderabad (IN); Prachi MANOREY, Indore (IN); Kumaraswamy DUNGA, Srikakulam (IN); Kishore Jahagirdar JAHAGIRDAR, Hyderabad (IN)

(21) Appl. No.: 18/852,717

(22) PCT Filed: Mar. 29, 2023
(86) PCT No.: PCT/IN2023/050300

§ 371 Date: Sep. 30, 2024

(30) Foreign Application Priority Data
Mar. 30, 2022 (IN) 202241018822

(51) Int. Cl. C07K 16/28; C07K 1/16; C07K 1/34

(52) U.S. Cl. CPC C07K 16/2818; C07K 1/16; C07K 1/34

(57) ABSTRACT

The method disclosed in the current invention is used to purify an antibody from process and product related impurities. The method discloses the use of affinity chromatography, cation exchange chromatography and mixed-mode chromatography for the reduction of process and product-related impurities. More specifically, the method discloses the use of cation exchange chromatography for the reduction of impurities such as high molecular weight aggregates and charge variants from an antibody composition.

### FIELD OF THE INVENTION

The present invention relates to protein purification methods. In particular, the invention relates to methods for purifying a composition comprising immunoglobulin using a combination of various chromatography steps.

### BACKGROUND

Monoclonal antibodies (mAbs) are effective targeted therapeutic agents. The high specificity of the antibodies makes them ideal to reach their intended target and hence is useful to treat a wide variety of diseases.

The commercial production of recombinant human monoclonal antibody therapeutics demands robust processes, i.e., the purification scheme needs to reliably and predictably produce an antibody composition intended for use in humans. A purification process should be designed to remove product related contaminants such as high molecular weight (HMW) aggregates, variants such as charge variants (acidic, deamidated/oxidized, basic), sequence variants and other variants, as well as process related contaminants such as leached Protein-A, host cell protein, DNA, adventitious and endogenous viruses, endotoxin, extractable from resins and filters, process buffers and agents such as detergents that may have been employed for virus reduction. In designing a purification scheme and other conditions for each of the chromatographic steps, along with removal of contaminants, an important consideration is recovery from each step of the purification scheme and from the overall purification scheme. Hence, for a commercially viable process, the purification scheme needs to be designed to ensure adequate removal of contaminants from an antibody composition while maintaining the yield of the same.

Product-related and process-related impurities, including aggregates and charge variants, have the potential to interfere with the purification process, affect the protein during storage, and/or can potentially be a cause of adverse reactions upon administration of an antibody to a subject as a pharmaceutical. Therefore, separation of the desired recombinant therapeutic protein from product- and process-related impurities to a purity sufficient for use as a human therapeutic poses a formidable challenge. Chromatographic techniques exploit the physical and chemical differences between the antibodies and the contaminant for the separation. Majority of purification schemes for mAbs involve a Protein-A based chromatography, which results in a high degree of purity and recovery in a single step. One or two additional chromatography steps are employed as polishing steps, generally selected from cation exchange chromatography, anion exchange chromatography, hydrophobic interaction chromatography or mixed-mode chromatography. The selection of the polishing chromatography steps is dictated majorly by the target antibody to be purified because every antibody is different in terms of its physico-chemical properties and may need a different purification scheme and/or different purification conditions for an efficient separation from impurities.

Removal of HMW aggregates, especially soluble aggregates, presents a challenge due to the physical and chemical similarity of the aggregates to the drug product itself, which is usually a monomer. In addition, the presence of unwanted charge variants such as far-basic variants pose additional challenges, which require additional efforts to fine-tune the purification scheme.

Hence, there is a need for an improved purification scheme to control the product- and process related species, including HMW aggregates and certain charge variants, in the final drug substance of a therapeutic antibody composition.

### SUMMARY

The present invention discloses a method for purifying an antibody composition comprising the target antibody and one or more contaminants, the method comprising the steps of affinity chromatography, cation exchange chromatography, and mixed-mode chromatography, wherein the cation exchange chromatography step comprises the steps of contacting the antibody composition with a negatively charged resin at an antibody concentration of less than about 40 grams per liter of the resin, wherein a significant amount of the target antibody binds to the resin, washing the resin with a wash buffer solution, and eluting the bound antibody with an elution buffer comprising citrate at pH about 6.0 and conductivity less than 10 mS/cm. The specific elution conditions employed effect up to 95% reduction of HMW aggregates, further maintaining the recovery of the antibody to be about 85% or more.

The method disclosed as per the current invention results in a significant reduction of HMW aggregates and the complete removal of a far-basic variant of the target antibody.

### DETAILED DESCRIPTION OF THE INVENTION

### Definitions

The phrase “ion exchange material” refers to a solid phase which is negatively charged (i.e., a cation exchange resin) or positively charged (i.e., an anion exchange resin). The charge may be provided by attaching one or more charged ligands to the solid phase, e.g. by covalent linking. Alternatively, or in addition, the charge may be an inherent property of the solid phase (e.g. as is the case for silica, which has an overall negative charge).

The term “conductivity” refers to the ability of an aqueous solution to conduct an electric current between two electrodes. In solution, the current flows by ion transport. Therefore, with an increasing amount of ions present in the aqueous solution, the solution will have a higher conductivity. The unit of measurement for conductivity is mS/cm, and can be measured using a conductivity meter, e.g., by Orion. The conductivity of a solution may be altered by changing the concentration of ions therein. For example, the concentration of a buffering agent and/or concentration of a salt (e.g. NaCl or KCl) in the solution may be altered in order to achieve the desired conductivity.

A “contaminant” is a material that is different from the desired polypeptide product. The contaminant may be a variant of the desired polypeptide (e.g. a deamidated variant or an aminoaspartate variant of the desired polypeptide) or another non-product related polypeptide, for e.g., host cell protein, host cell nucleic acid, endotoxin, etc. A contaminant can also be process related, for example—Protein-A-leachates.

“High molecular weight aggregates” as referred herein encompasses association of at least two molecules of a product of interest, e.g., antibody or any antigen-binding fragment thereof. The association of at least two molecules of a product of interest may arise by any means including, but not limited to, non-covalent interactions such as, e.g., charge-charge, hydrophobic and van der Waals interactions; and covalent interactions such as, e.g., disulfide interaction or non-reducible crosslinking. An aggregate can be a dimer, trimer, tetramer, or a multimer greater than a tetramer, etc.

The term “process or product related impurities” as used herein refer to the contaminants which may be derived from the manufacturing process, for example, but not limited to, cell culture, downstream or cell substrates and may include host cell proteins, host cell DNA, nucleic acid, protein-A leachates etc., or may be molecular variants of the protein of interest, for example, but not limited to, high molecular weight (HMW) aggregates, acidic variants, basic variants, low molecular weight variants etc., and may be formed during expression, manufacture or storage of the protein.

The term ‘variants’ as used here in refers to a group of low-pI, mid-pI and high-pI variants, and are described as “acidic variants”, “mid peak” and “basic variants” respectively, based on their differential elution from an analytical ion-exchange HPLC.

An ‘acidic variant’ is a variant of a polypeptide of interest which is more acidic than the polypeptide of interest. An acidic variant species elute earlier than the main peak when determined by a standard cation exchange chromatography.

A ‘basic variant’ is a variant of a polypeptide of interest which is more basic than the polypeptide of interest. A basic variant species elute later than the main peak when determined by a standard cation exchange chromatography. A ‘far basic variant’ is a variant that is more basic than the basic variant of a polypeptide of interest. A far basic variant species elute later than the basic variant when determined by a standard cation exchange chromatography.

The term “about” as used herein, means an acceptable error range for the particular value as determined by one of ordinary skill in the art. For example, “about” can mean a range of up to 20%.

The “composition” to be purified herein comprises the protein of interest and one or more contaminants. The composition may be “partially purified” (i.e., having been subjected to one or more purification steps) or may be obtained directly from a host cell or organism producing the antibody (e.g., the composition may comprise harvested cell culture fluid).

The term “load” herein refers to the composition loaded onto the chromatography material, i.e., ion exchange support. Preferably, the chromatography material is equilibrated with an equilibration buffer prior to loading the composition which is to be purified.

The term “bind and elute mode” as used herein refers to a process wherein the target protein substantially binds to the chromatographic support, and is subsequently eluted from the chromatographic support.

The term “Mixed-Mode Chromatography” refers to a form of chromatography that uses a chromatographic support with at least two unique types of functional groups, each interacting with the molecule or protein of interest. Mixed-mode chromatography generally uses ligands that have more than one type of interaction with target proteins and/or impurities. For example, a charge-charge type of interaction and/or a hydrophobic or hydrophilic type of interaction, or an electroreceptor-donor type interaction. In general, based on the difference in the total interaction, the target protein and one or more impurities can be separated under various conditions.

Aggregate concentration can be measured in a protein sample using Size Exclusion Chromatography (SEC), a well-known and widely accepted method in the art. Size exclusion chromatography uses a molecular sieving retention mechanism, based on differences in the hydrodynamic radii or differences in size of proteins. Large molecular weight aggregates cannot penetrate or only partially penetrate the pores of the stationary phase. Hence, the larger aggregates elute first and smaller molecules elute later, the order of elution being a function of the size.

### DETAILED DESCRIPTION OF THE EMBODIMENTS

The present invention discloses a method to purify an antibody composition comprising the target antibody and one or more contaminants, for example, high molecular weight aggregates, host cell proteins/nucleic acids, protein-A leachates, and charge variants, the method comprises the use of a combination of affinity chromatography, cation exchange chromatography and mixed-mode chromatography.

In an embodiment, the method disclosed herein is used to reduce the level of process and product related impurities in an antibody composition comprising an anti-PD1 antibody and one or more said impurities using a combination of affinity chromatography, cation exchange chromatography and mixed-mode chromatography.

In another embodiment, the method disclosed herein is used to reduce the level of process and product related impurities in an antibody composition comprising an anti-PD1 antibody and one or more said impurities, wherein the method comprises the following steps:
(a) cell culture clarification
(b) affinity chromatography
(c) low-pH viral inactivation and pH neutralization
(d) depth filtration
(e) cation exchange chromatography
(f) mixed-mode chromatography
(g) nano-filtration
(h) ultrafiltration/diafiltration

In another embodiment, the method disclosed herein is used to reduce the level of process and product related impurities in an antibody composition comprising nivolumab and one or more said impurities, wherein the method comprises the following steps:
(a) cell culture clarification
(b) affinity chromatography
(c) low-pH viral inactivation and pH neutralization
(d) depth filtration
(e) cation exchange chromatography
(f) mixed-mode chromatography
(g) nano-filtration
(h) ultrafiltration/diafiltration

In yet another embodiment, the method disclosed herein is used to reduce the level of impurities such as HMW aggregates and far-basic variants in an antibody composition comprising an anti-PD1 antibody and one or more said impurities using cation exchange chromatography, wherein the antibody composition is loaded onto the cation exchange support in the presence of a loading buffer solution under such conditions that the target antibody substantially binds to the cation exchange support, and the bound antibody is eluted from the cation exchange support by a buffer solution comprising citrate, and wherein the cation exchange support is washed using a wash buffer only once between the loading and the elution steps.

In yet another embodiment, the method disclosed herein is used to reduce the level of impurities such as HMW aggregates and far-basic variants in an antibody composition comprising an anti-PD1 antibody and one or more said impurities, the method comprising steps of:
(a) loading the antibody composition onto a cation exchange support in the presence of a loading buffer solution under conditions such that the anti-PD1 antibody substantially binds to the cation exchange support,
(b) washing the cation exchange support with a wash buffer solution,
(c) eluting the bound antibody using an elution buffer, and
(d) collecting the eluate from the cation exchange support,
wherein the elution buffer solution comprises citrate,
and wherein the cation exchange support is washed using a wash buffer only once between the loading and the elution steps.

In a further embodiment, the method disclosed herein is used to reduce the level of impurities such as HMW aggregates and far-basic variants in an antibody composition comprising an anti-PD1 antibody and one or more said impurities, the method comprising steps of:
(a) loading the antibody composition onto a cation exchange support in the presence of a loading buffer solution under conditions such that the anti-PD1 antibody substantially binds to the cation exchange support,
(b) washing the cation exchange support with a wash buffer solution,
(c) eluting the bound antibody using an elution buffer, and
(d) collecting the eluate from the cation exchange support,
wherein the elution buffer solution comprises citrate, has a pH of about 6.0 and conductivity less than 10 mS/cm,
and wherein the cation exchange support is washed using a wash buffer only once between the loading and the elution steps.

In a further embodiment, the method disclosed herein is used to reduce the level of impurities such as HMW aggregates and far-basic variants in an antibody composition comprising nivolumab and one or more said impurities, the method comprising steps of:
(a) loading the antibody composition onto a cation exchange support in the presence of a loading buffer solution under conditions such that nivolumab substantially binds to the cation exchange support,
(b) washing the cation exchange support with a wash buffer solution,
(c) eluting the bound antibody using an elution buffer, and
(d) collecting the eluate from the cation exchange support, wherein the elution buffer solution comprises citrate, and wherein the cation exchange support is washed using a wash buffer only once between the loading and the elution steps.

In a further embodiment, the method disclosed herein is used to reduce the level of impurities such as HMW aggregates and far-basic variants in an antibody composition comprising nivolumab and one or more said impurities, the method comprising steps of:
(a) loading the antibody composition onto a cation exchange support in the presence of a loading buffer solution under conditions such that nivolumab substantially binds to the cation exchange support,
(b) washing the cation exchange support with a wash buffer solution,
(c) eluting the bound antibody using an elution buffer, and
(d) collecting the eluate from the cation exchange support,
wherein the elution buffer solution comprises citrate, has a pH of about 6.0 and conductivity less than 10 mS/cm,
and wherein the cation exchange support is washed using a wash buffer only once between the loading and the elution steps.

In any of the above mentioned embodiments, the high molecular weight aggregates are reduced by up to 95% in the eluate collected from the cation exchange support as compared to the level of high molecular weight aggregates in the antibody composition loaded onto the cation exchange support.

In any of the above mentioned embodiments, the method results in complete removal of the far-basic variant of the antibody.

In any of the above mentioned embodiments, the method is also used to reduce the level of other process-related impurities, including but not limited to protein-A leachates, host cell proteins, host cell DNA, etc.

In any of the above mentioned embodiments, the CEX is operated in bind and elute mode.

In any of the above mentioned embodiments, the antibody is an anti-PD-1 antibody or antigen binding fragment thereof.

In any of the above mentioned embodiments, the antibody is nivolumab.

The invention is more fully understood by reference to the following examples. These examples should not, however, be construed as limiting the scope of the invention.

### EXAMPLES

### Example 1

A therapeutic monoclonal antibody which binds and blocks programmed death receptor-1 (PD-1) was cloned and expressed in a Chinese Hamster Ovary cell line and the cell culture broth containing the expressed antibody was harvested, clarified and subjected to protein-A affinity chromatography. The eluate from protein-A affinity chromatography was subjected to low-pH incubation and depth filtration, and the filtered liquid comprising the antibody composition was further purified using cation exchange chromatography (CEX). Level of impurities was determined in both load and eluate of CEX. Details of CEX chromatography are given in Tables 1 and 2.

TABLE 1
Chromatography conditions used in CEX

| Resin | Poros ™ XS |
|---|---|
| Bed Height (cm) | 18-22 |
| Residence Time (min) (loading, post- | 4.0 |
| load wash and elution) | |
| Load Factor (g/L) | <40 |

TABLE 2
Details of buffers used in CEX

| | | | Conductivity |
|---|---|---|---|
| Stage | Buffer | pH | (mS/cm) |
| Equilibration | 10 mM sodium citrate buffer | 6.0 ± 0.2 | 4.2 ± 0.5 |
| Wash | 10 mM sodium citrate + | 6.0 ± 0.2 | 4.2 ± 0.5 |
| | 20 mM NaCl | | |
| Elution | 10 mM sodium citrate + | 6.0 ± 0.2 | 9.2 ± 1.0 |
| | 70 mM NaCl | | |
| Regeneration | 0.5 M NaOH | NA | NA |

Table 3 summarizes the HMW aggregate level at the time of loading onto CEX and in the eluate obtained from CEX at elution buffer pH of 5.9.

TABLE 3
HMW aggregate level in CEX load and CEX eluate

| | HMW % | HMW % | % | % |
|---|---|---|---|---|
| Batch | (load) | (eluate) | Reduction | Recovery |
| Nmab-1 | 6.7 | 0.3 | 95.5 | 88 |
| Nmab-2 | 4.6 | 0.4 | 91.3 | 83 |
| Nmab-3 | 1.4 | 0.3 | 78.6 | 90 |
| Nmab-4 | 1.4 | 0.3 | 78.6 | 89 |

| | | |
|---|---|---|
| Average | 86.0 | 87.5 |

Similarly, the levels of far-basic variants were determined in CEX load and eluate and are represented in Table 4 along with HCD content in CEX eluate.

TABLE 4
Far-basic variant levels at CEX load and CEX eluate stages

| | Far Basic | Far Basic | |
|---|---|---|---|
| | variant % | variant % | % |
| Batch | (load) | (eluate) | Reduction |
| Nmab-1 | 6.9 | 0 | 100% |
| Nmab-2 | 7.9 | 0 | 100% |
| Nmab-3 | 9.0 | 0 | 100% |
| Nmab-4 | 4.9 | 0 | 100% |

It is evident from Tables 3 and 4 that the disclosed method is able to significantly reduce the levels of HMW aggregates and completely removes the far-basic variants from the antibody composition.

1. A method to reduce the level of high molecular weight aggregates and far-basic variants impurities content in an antibody composition, comprising an anti-PD1 antibody and one or more said impurities, using cation exchange chromatography wherein the antibody composition is loaded onto the cation exchange support in the presence of a loading buffer solution under such conditions that the target antibody substantially binds to the cation exchange support, and the bound antibody is eluted from the cation exchange support by a buffer solution comprising citrate, and wherein the cation exchange support is washed using a wash buffer only once between the loading and the elution steps.

2. A method disclosed to reduce the level of high molecular weight aggregates and far-basic variants impurities content in an antibody composition, comprising an anti-PD1 antibody and one or more said impurities, the method comprising steps of:
(a) loading the antibody composition onto a cation exchange support in the presence of a loading buffer solution under conditions such that the anti-PD1 antibody substantially binds to the cation exchange support,
(b) washing the cation exchange support with a wash buffer solution,
(c) eluting the bound antibody using an elution buffer, and
(d) collecting the eluate from the cation exchange support,
wherein the elution buffer solution comprises citrate, has a pH of about 6.0 and conductivity less than 10 mS/cm,
and wherein the cation exchange support is washed using a wash buffer only once between the loading and the elution steps.

3. The method according to claim 1, wherein the high molecular weight aggregates are reduced by up to 95% in the eluate collected from the cation exchange support as compared to the level of high molecular weight aggregates in the antibody composition loaded onto the cation exchange support.

4. The method according to claim 1, wherein the far-basic variants of the antibody are completely removed/100% removed in the eluate collected from the cation exchange support.

5. The method according to claim 1, wherein the method is preceded by an affinity chromatography step.

6. The method according to claim 1, wherein the method comprises a mixed mode chromatography step.

7. The method according to claim 1, further comprises one or more filtration steps.

8. The anti-PD1 antibody according to claim 1, is nivolumab.

9. The method according to claim 2, wherein the high molecular weight aggregates are reduced by up to 95% in the eluate collected from the cation exchange support as compared to the level of high molecular weight aggregates in the antibody composition loaded onto the cation exchange support.

10. The method according to claim 2, wherein the far-basic variants of the antibody are completely removed/100% removed in the eluate collected from the cation exchange support.

11. The method according to claim 2, wherein the method is preceded by an affinity chromatography step.

12. The method according to claim 2, wherein the method comprises a mixed mode chromatography step.

13. The method according to claim 2, further comprises one or more filtration steps.
